(19) United States
(12) Patent Application Publication
(10) Pub. No.: US 2023/0210862 A1
(43) Pub. Date: Jul. 6, 2023

(54) FORMULATION AND METHOD FOR TREATING PROSTATE CANCER

(71) Applicant: INDIAN INSTITUTE OF TECHNOLOGY KANPUR, KANPUR (IN)

(72) Inventors: Bushra Ateeq, KANPUR (IN); Sakshi Goel, KANPUR (IN)

(21) Appl. No.: 18/147,030

(22) Filed: Dec. 28, 2022

Related U.S. Application Data
(60) Provisional application No. 63/295,018, filed on Dec. 30, 2021.

(51) Int. Cl. A61K 31/551; A61K 31/4166; A61P 13/08; A61P 35/00

(52) U.S. Cl. CPC A61K 31/551; A61K 31/4166; A61P 13/08; A61P 35/00

(57) ABSTRACT

A formulation and method for treating prostate cancer is provided. The method includes use of bromodomain and extra-terminal domain inhibitors (BETi) or combination of BETi and anti-androgen drug to therapeutically target DLX1-positive advanced-stage prostate cancer patients. The formulation for treating prostate cancer relates to disrupting ERG/AR transcriptional circuitry with BETi in combination with anti-androgen drug to attenuate DLX1 expression and its downstream oncogenic effects. The BETi and the combination of BETi and anti-androgen drug yields 60% of tumor regression and remarkable reduction in distant metastases in the preclinical immunodeficient mice bearing DLX1-positive tumors.

### EARLIEST PRIORITY DATE

This application claims priority from a Provisional patent application filed in the United States of America having Patent Application No. 63/295,018, filed on Dec. 30, 2021 sand titled “A METHOD FOR TREATING PROSTATE CANCER”.

### FIELD OF INVENTION

Embodiments of a present disclosure relate to therapeutic intervention for the treatment of cancer and more particularly to formulations and method for treating prostate cancer.

### BACKGROUND

Prostate cancer (PCa) is the second most frequently diagnosed cancer and has fifth highest mortality rate in men, worldwide. The prostate cancer experienced ˜1.27 million new cases and 358,989 deaths accounting for 3.8% of total deaths in men caused by cancer in 2018. According to Cancer Statistics, Surveillance, Epidemiology, and End Results Program (SEER), PCa is frequently diagnosed among men with median age of 67. The PCa is a heterogeneous disease and is classified into various molecular subtype involving gene rearrangements between androgen-regulated transmembrane protease Serine 2 (TMPRSS2) and erythroblast transformation-specific (ETS) transcription factor, v-ets erythroblastosis virus E26 oncogene homolog (ERG), recurrent in −50% of the PCa patients. TMPRSS2-ERG gene fusion results in aberrant overexpression of ERG which controls a transcriptional network linked to PCa development, increased metastatic potential, and associate with poor clinical outcome.

Considering the critical role of androgen receptor (AR) signaling in the development and progression of PCa, inhibitors targeting androgen synthesis (abiraterone acetate) and AR antagonists (bicalutamide, enzalutamide) are used in the first-line of treatment termed as androgen deprivation therapy (ADT), often administered to advanced stage PCa patients. Although, prolonged administration of ADT results in an inevitable cancer relapse due to the selection pressure of drugs, eventually progressing to aggressive castration-resistant prostate cancer (CRPC) stage. Mounting evidence suggests sustained AR activity in CRPC, owing to numerous mechanisms including AR amplification, gene mutations, intra-tumoral androgen synthesis, and expression of constitutively active AR splice variants (AR-Vs). AR-V7 lacks the ligand binding domain and hence, functions in a ligand-independent manner resulting in constitutively active AR. The AR-V7 is responsible for driving androgen-independent growth of the PCa thereby contributing in CRPC progression. Bromodomain and Extra-Terminal (BET) proteins are the known transcriptional coactivators of tumor promoting genes such as AR and ERG and are considered as a potential therapeutic target for CRPC treatment. Inhibitors against BET (BETi) proteins attenuate AR and ERG-mediated oncogenesis in CRPC by disrupting transcriptional activation complex at their target gene loci. of late, several BETi including OTX-015, ZEN003694, and GS-5829 are in clinical trials as single agents or in combination with anti-androgens for CRPC patients.

Deregulation of homeobox genes has been linked to several human malignancies including prostate. A Distal-less homeobox-1 gene (DLX1) a member of the DLX homeobox family plays an essential role in the development of craniofacial features, jaw, and GABAergic (gamma-aminobutyric acid) intemeuron. In the PCa, DLX1 has been validated as a PCa biomarker across clinically independent cancer cohorts, wherein DLX1 and HOXC6 accurately predict high-grade disease. However, the underlying regulatory mechanism that drives DLX1 upregulation remain poorly understood, hence identification of the transcriptional regulatory circuitry would assist the therapeutic targeting of DLX1-positive subset of prostate cancer patients.

Hence, there is a need for formulations and an effective method for treating prostate cancer targeting the DLX1-positive subset of prostate cancer patients.

### SUMMARY

In accordance with an embodiment of the present invention, a formulation for treating prostate cancer is provided. The formulation includes at least one of bromodomain and extra-terminal domain inhibitors (BETi) and an anti-androgen drug or combination thereof. The formulation is configured to disrupt E26 oncogene homolog and androgen receptor (ERG/AR) transcriptional circuitry to attenuate Distal-less homeobox gene 1 (DLX1) mediated tumorigenesis. The formulation is also configured to reduce DLX1 expression and downstream target genes of DLX1 in transmembrane protease Serine 2 and ERG (TMPRSS2-ERG) fusion positive prostate cancer cells and TMPRSS2-ERG fusion negative prostate cancer cells.

In accordance with another embodiment of the present invention, a method for treating prostate cancer is provided. The method includes administering a therapeutically effective amount of a formulation to a subject with prostate cancer. The formulation comprises at least one of bromodomain and extra-terminal domain inhibitors (BETi) and an anti-androgen drug or combination thereof. The formulation is configured to disrupt E26 oncogene homolog and androgen receptor (ERG/AR) transcriptional circuitry to attenuate Distal-less homeobox gene 1 (DLX1) mediated tumorigenesis. The formulation is also configured to reduce DLX1 expression and downstream target genes of DLX1 in transmembrane protease Serine 2 and ERG (TMPRSS2-ERG) fusion positive prostate cancer cells and TMPRSS2-ERG fusion negative prostate cancer cells. The BETi and the combination of BETi and anti-androgen drug yields 60% of tumor regression in immunodeficient mice.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will be described and explained with additional specificity and detail with the accompanying figures in which:

FIG. 1 represents a chart of dosage, route, and frequency of administrating the formulation and a vehicle control, in accordance with an embodiment of the present disclosure;

FIG. 2 illustrates upregulation of DLX1 in prostate cancer (PCa) A) Dot plot showing DLX1 expression in TCGA-PRAD RNA-Seq dataset, data represents log 2 (norm_count+1) and whiskers (error bars) denotes standard deviations (SD); B) Dot plot of DLX1 expression using microarray profiling data (GSE35988), log 2 (norm_mRNA), and the error bar represents SD; C) Kaplan-Meier plot showing survival probability in TCGA-PRAD (n=498) dataset categorized in high DLX1 (DLX1^{Hi}) and low DLX1 (DLX1^{Lo}) expression; and D) Q-PCR data showing DLX1 expression in a panel of PCa cell lines, in accordance with an embodiment of the present disclosure;

FIG. 3 illustrates characterization of DLX1 overexpression and knockout cells A) Q-PCR data showing relative expression of DLX1 in isogenic RWPE1 cells overexpressing DLX1; B) Immunoblot showing protein level of DLX1 in cells same as A); C) Q-PCR showing DLX1 expression in 22RV1-DLX1-KO and 22RV1-SCR control cells; and D) Immunoblot showing protein level of DLX1 in cells same as C), in accordance with an embodiment of the present disclosure;

FIG. 4 illustrates DLX1 regulates oncogenic properties in prostate cells A) cell proliferation assay using isogenic RWPE1 cells overexpressing DLX1 at indicated time-points; B) foci formation assay using same cells as A); C) Boyden Chamber Matrigel migration assay using same cells as A); D) cell proliferation assay using 22RV1-DLX1-KO (C-1, C-2 and C-3 are independent clones) and control cells at indicated time-points; E) Boyden Chamber Matrigel migration assay using same cells as D); and F) Anchorage-independent soft agar assay using same cells as D), in accordance with an embodiment of the present disclosure;

FIG. 5 illustrates regulation of tumor associated biological processes by DLX1 A) DAVID analysis showing unregulated (right) and downregulated (left) biological processes in 22RV1-DLX1-KO against control cells, bars represent the −log 10 (P-value) and the frequency polygon (black line) denotes number of genes; and B) gene set enrichment analysis (GSEA) plots representing deregulated pathways, in accordance with an embodiment of the present disclosure;

FIG. 6 illustrates genetic ablation of DLX1 orchestrates cancer hallmarks A) Q-PCR data showing expression of EMT markers in 22RV1-DLX1-KO and control SCR cells; B) immunoblots showing vimentin and E-cadherin using same cells as A), β-actin is used as loading control; C) immunoblots showing vimentin and E-cadherin same as B) except for cleaved PARP, cleaved Caspase-3 and Bcl-xL; D) Q-PCR data for stem cell markers using 22RV1-DLX1-KO and control SCR cells; and E) fluorescence intensity of catalyzed ALDH substrate in 22RV1-DLX1-KO and control cells, marked windows show ALDH1+ percent cell population, in accordance with an embodiment of the present disclosure;

FIG. 7 illustrates DLX1 expression abrogating results in tumor regression and reduced metastases A) mean tumor volume of 22RV1-DLX1-KO and control SCR cells subcutaneously implanted in NOD/SCID mice (n=6); B) representative images of the tumors excised at end of the xenograft experiment (top panel), bar graph showing relative percent reduction in tumor burden (bottom panel); C) bar graphs representing number of cells metastasized to the bone marrow in xenografted mice as labelled (n=5); and D) bar graphs representing number of cells metastasized to the lungs in xenografted mice as labelled (n=5), in accordance with an embodiment of the present disclosure;

FIG. 8 illustrates regulation of DLX1 attenuation proliferation, EMT and stemness markers A) images depicting immunostaining for Ki-67, E-cadherin (E-Cad), Vimentin (VIM), and ALDH1A1 on xenograft tumor sections; B) box plots showing immunostaining quantification of Ki-67, E-Cad, VIM and ALDH1A1, in accordance with an embodiment of the present disclosure;

FIG. 9 illustrates regulation of the bone metastatic potential of PCa cells by DLX1 A) representative microCT bone images showing horizontal section (top panel) and vertical cross-section (bottom panel) views of the tibia excised from mice (n=6), four weeks after intra-medullary tibia injection using 22RV1-DLX1-KO and control cells; B) representative microCT bone images same as A) except box plots showing bone architecture parameters analyzed using CTAn software, in accordance with an embodiment of the present disclosure;

FIG. 10 illustrates correlation of elevated ERG and AR expression with higher DLX1 levels A) representative core of the PCa tissue microarray (TMA) showing RNA in-situ hybridization (RNA-ISH) scoring pattern for DLX1 in 144 PCa patient specimens; B) bar plot showing percentage of patients negative (DLX1−) and positive (DLX1+) for DLX1 expression based on the scoring pattern; C) representative core of the PCa TMA same as A) except Immunohistochemistry (IHC) for ERG (top panel) and RNA-ISH for DLX1 (bottom) in 144 PCa patient tissue specimens; D) contingency table depicting status of DLX1 and ERG; E) bar plot showing association between ERG and DLX1 expression status and Gleason scores of PCa patients; F) representative core of the PCa TMA same as A) except representative tumor cores showing IHC for AR, ERG and RNA-ISH for DLX1 representing AR+/ERG+/DLX1+ status in 144 PCa patient tissue specimens; G) same as F) except for representative AR+/ERG−/DLX1+ patient in TMA containing 144 PCa specimens; H) contingency table for the AR and DLX1 status in TMA patient specimens; and I) bar plot showing association between DLX1 expression and Gleason scores (left panel) of tumor specimens, and association of DLX1 expression with tumor stage (right panel), in accordance with an embodiment of the present disclosure;

FIG. 11 illustrates higher level of DLX1 in metastatic CRPC patients A) Heatmap showing DLX1 levels in tumor specimens representing distant metastatic sites of metastatic CRPC patients; B) Heatmap showing DLX1 levels same as A) except for RNA-ISH for DLX1 expression in TMA containing 121 mCRPC biospecimens collected from various metastatic sites; and (C) bar plot showing DLX1 expression in percent metastatic sites from CRPC patients same as A), in accordance with an embodiment of the present disclosure;

FIG. 12 illustrates DLX1 is a transcriptional target of ERG and/or AR in PCa A) schema showing chromosomal location of the ERG binding motifs (EBM) onto DLX1 promoter selected for ChIP-qPCR (top panel). ChIP-qPCR data showing recruitment of ERG at the DLX1 promoters; B) Q-PCR data showing the expression of ERG and DLX1 in RWPE1 cells overexpressing ERG; and C) schema showing site-directed mutagenesis of DLX1 promoter cloned upstream of luciferase gene, nucleotides in red are mutated (top), in accordance with an embodiment of the present disclosure;

FIG. 13 illustrates regulation of DLX1 expression in TMPRSS2-ERG fusion positive PCa by coordinating ERG, AR and FOXA1 coordinates A) schematic showing the androgen response elements (AREs) at the DLX1 putative enhancer (top panel); B) ChIP-qPCR data depicting FOXA1 recruitment at the DLX1 putative enhancer in R1881 (10 nM) stimulated VCaP; C) integrated genome view of 3D chromatin structure and binding of transcription factors at the genomic and nearby region of DLX1; D) Q-PCR data showing relative expression of DLX1 in siRNA-mediated ERG, AR and/or FOXA1 silenced VCaP cells; and E) same as D) except immunoblot data, in accordance with an embodiment of the present disclosure;

FIG. 14 illustrates regulation of transcriptional regulation of DLX1 in TMPRSS2-ERG fusion positive PCa by AR and FOXA1 A) ChIP-qPCR data depicting AR (top panel) and FOXA1 (bottom panel) enrichment at the DLX1 putative enhancer in 22RV1 cells stimulated with R1881 (10 nM) for 16 hours; B) relative expression of KLK3 and DLX1 in doxycycline (Dox) induced AR-V7 overexpressing LNCaP cells treated with R1881 (10 nM); C) immunoblots showing the expression of AR-FL, AR-V7, DLX1 and PSA using same cells as B); D) Q-PCR data showing DLX1 and KLK3 expression in LNCaP AR-V7 cells under similar culture conditions as mentioned at 48-hour time point; in accordance with an embodiment of the present disclosure;

FIG. 15 illustrates downregulation of DLX1 expression by BET inhibitor alone or in combination with anti-androgen A) Q-PCR data showing relative expression of DLX1, KLK3 and DLX1 target genes, namely, HNF1A and ALDH1A1 in VCaP cells treated with Enza (10 μM), JQ1 (0.5 μM) alone or in combination for 48 hours; B) Q-PCR data same as A) except immunoblot; C) Q-PCR data same as A) except 22RV1 cells; and D) Q-PCR data same as C) except immunoblot, in accordance with an embodiment of the present disclosure;

FIG. 16 illustrates BET inhibitor or/and anti-androgen orchestrates DLX1 mediated oncogenic properties A) cell proliferation assay in VCaP cells treated Enza (10 μM), JQ1 (0.5 μM) alone or in combination for 48 hours; B) cell proliferation assay same as A) except 22RV1 cells; C) Boyden Chamber Matrigel migration assay in VCaP cells using same treatment conditions as A); D) Boyden Chamber Matrigel migration assay same as C) except 22RV1 cells; E) Fluorescence intensity of the catalyzed ALDH substrate in VCaP cells under same treatment conditions as A); and F) Fluorescence intensity same as E) except 22RV1 cells, in accordance with an embodiment of the present disclosure;

FIG. 17 illustrates attenuation of DLX1-mediated tumorigenesis and metastases by BET inhibitor alone or in combination with anti-androgen A) mean tumor volume of xenografts generated by implanting 22RV1 cells in athymic nude mice, and randomized into four treatment groups (n=6 each), namely, vehicle control, Enza (20 mg/kg), JQ1 (50 mg/kg), and a combination of Enza and JQ1; B) bar plot showing percent tumor reduction in the treatment groups (n=6) compared with vehicle control group; C) mean body weight of mice during treatment with drugs as mentioned in A); D) scatter dot plot showing number of cells metastasized to bone marrow in xenografted mice treated with drugs as mentioned in A); and E) scatter dot plot same as D) except for showing number of cells metastasized to lungs, in accordance with an embodiment of the present disclosure; and

FIG. 18 illustrates reduction in expression of DLX1 by BET inhibitor or/and anti-androgen, proliferative and stem-cell marker A) representative images depicting IHC staining for Ki-67 (proliferation marker), ALDH1A1 (DLX1 target gene), and RNA-ISH for DLX1 using formalin-fixed paraffin-embedded tumor xenograft specimens of nude mice administered with vehicle control, Enza (20 mg/kg), JQ1 (50 mg/kg), and a combination of Enza and JQ1 (n=5 per group); and B) box plots showing quantification of Ki-67, ALDH1A1 and DLX1 expression in the tumor tissue sections (n=5) of the mice xenografts as A), in accordance with an embodiment of the present disclosure.

Further, those skilled in the art will appreciate that elements in the figures are illustrated for simplicity and may not have necessarily been drawn to scale. Furthermore, in terms of the method steps, chemical compounds, and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

As used herein, the term “cancer” or “tumor” describes the physiological condition in mammals that is characterized by the uncontrolled cell growth. The terms “oncogenic”, “tumorigenic”, “oncogenicity” or related terms to that of cancer, describes the properties of the cells which are specific to cancer.

As used herein, the term “genetic ablation” refers to the deletion of genomic region of a gene that codes for functional protein. Hence, genetic ablation results in removal of the gene expression by genetically modifying the chromosomal region in a host cell.

As used herein, the term “tumor tissue” refers to tumor formed in the mice implanted with PCa cells in a laboratory setup.

As used herein, the term “patient”, “subject” or “specimen” refers to any single subject or the excised tumor tissue which is included in the study. In the referred embodiments subject is a male human suffering from prostate cancer with the age between 45-90 years. In the referred embodiments, subject is a preclinical mice model suffering from prostate cancer.

As used herein, the term “tumor burden” refers to the size of the tumor measured using digital Vernier caliper and the tumor volume was calculated using the length and width of a tumor measured.

As used herein, the term “administration” or “treatment” refers to the contact of an animal, human, experimental subject, cell or the fluid in which cells are cultured, with the exogenous pharmaceutical or therapeutic agent or composition. It also refers to the in vitro and in vivo treatments.

Embodiments of the present invention relates to a formulation and method for treating prostate cancer (PCa). The invention mainly focuses on reduction of a Distal-less homeobox-1 gene (DLX1) expression, its downstream target genes and oncogenic properties including cell proliferation and migration in order to mitigate the prostate cancer.

This research was supported by the Science and Engineering Research Board (SERB), Government of India (EMR/2016/005273 to B.A.).

In an embodiment, a formulation for treating prostate cancer is provided. The formulation includes at least one of bromodomain and extra-terminal domain inhibitors (BETi) and an anti-androgen drug or combination thereof. The BETi includes but not limited to JQ1. The JQ1 is selected from a group consisting of inhibitors against BRD4, BRD2, BRD2/4 and a combination thereof. The anti-androgen drug includes but not limited to Enzalutamide.

In one embodiment, the formulation includes bromodomain and extra-terminal domain inhibitors (BETi). The formulation comprises 50 mg/kg of the BETi in treating DLX1-positive prostate cancer. The amount of BETi is 50 mg/kg of body weight of a subject. In one embodiment, the subject is the preclinical mice model. The BETi yields 60% of tumor regression in immunodeficient mice bearing DLX1 positive tumor. The formulation depends on the route of administration, severity of the disease and the pharmaceutical composition. For example, the formulation of BETi may vary between 20 mg/kg to 50 mg/kg in the mice model of different cancer types. In one embodiment, 50 mg/kg (50 mg compound/kg body weight of mice) of the BETi dosage is injected intraperitoneally 5 days a week for total duration of 4 weeks which imparted the best results in treating DLX1 positive prostate cancer.

In one embodiment, the formulation includes a combination of BETi and anti-androgen drug. The formulation comprises 50 mg/kg of BETi in combination with 20 mg/kg of anti-androgen drug in treating DLX1-positive prostate cancer. The amount of the BETi and the anti-androgen drug are per kg body weight of the subject i.e., the preclinical mice model. The combination of BETi and anti-androgen drug yields 60% of tumor regression in immunodeficient mice bearing DLX1 positive tumor. The formulation depends on the route of administration, severity of the disease and the pharmaceutical composition. For example, in one embodiment, 50 mg/kg of the BETi dosage is injected intraperitoneally and 20 mg/kg (20 mg compound/kg body weight of mice) of the anti-androgen is administered orally 5 days a week for total duration of 4 weeks which imparted the best results in treating DLX1 positive prostate cancer.

In the present invention, to examine efficacy of the formulation (drugs) in preclinical mice model, the formulation is administered to immunodeficient mice bearing DLX1 positive tumors to reduce burden of cancer cells. The pharmaceutical composition may refer to diluents, salts, solvents, carriers or vehicles which are suitable to retain an active form of the formulation and selected depending on the mode of administration. For example, compositions described in some of the embodiments include but not limited to polyethylene glycol 400 (PEG-400), corn oil, dimethyl sulfoxide (DMSO), cyclodextrin and water. The formulation may constitute 1-95% of the total composition weight. The Enzalutamide (anti-androgen) is diluted in 5% DMSO, 30% PEG-400 and 65% corn oil, while JQ1 (BETi) is diluted in 10% cyclodextrin in water.

Route of administration of the formulation may be oral or parenteral (intravenous, intramuscular, intraperitoneal and other mode of administrations). The embodiment in the present invention includes but not limited to the oral administration for anti-androgen drug and intraperitoneal injection (i.p.) for BETi. In case of combinatorial treatment both the formulations may be included in a single composition by selecting an appropriate pharmaceutical acceptable amount or can be administered separately. The formulation is configured to disrupt E26 oncogene homolog and androgen receptor (ERG/AR) transcriptional circuitry to attenuate Distal-less homeobox gene 1 (DLX1) mediated tumorigenesis. The formulation is also configured to reduce DLX1 expression and downstream target genes of DLX1 in transmembrane protease Serine 2 and ERG (TMPRSS2-ERG) fusion positive prostate cancer cells and TMPRSS2-ERG fusion negative prostate cancer cells.

In an exemplary embodiment, a set of formulations for treating prostate cancer is provided. The set of formulations includes a first formulation and a second formulation. The first formulation comprises bromodomain and extra-terminal domain inhibitors (BETi). The BETi is selected from a group consisting of BRD4, BRD2, BRD2/4 and combination thereof. The second formulation comprises combination of BETi and anti-androgen drug. The anti-androgen drug comprises Enzalutamide.

In another embodiment of the present invention, a method for treating prostate cancer is provided.

The method for treating prostate cancer begins with administering a therapeutically effective amount of a formulation to a subject with prostate cancer at step 102. The formulation includes at least one of bromodomain and extra-terminal domain inhibitors (BETi) and an anti-androgen drug or combination thereof. The BETi includes but not limited to JQ1. The JQ1 is selected from a group consisting of inhibitors against BRD4, BRD2, BRD2/4 and a combination thereof. The anti-androgen drug includes but not limited to Enzalutamide.

In one embodiment, the formulation includes bromodomain and extra-terminal domain inhibitors (BETi). The formulation includes 50 mg/kg of the BETi in treating DLX1-positive prostate cancer. The BETi yields 60% of tumor regression in immunodeficient mice bearing DLX1 positive tumor. In an exemplary embodiment, the formulation is administered in tumor-bearing immunodeficient mice. In one embodiment, the formulation includes a combination of BETi and anti-androgen drug. The formulation comprises 50 mg/kg of BETi in combination with 20 mg/kg of anti-androgen drug in treating DLX1-positive prostate cancer.

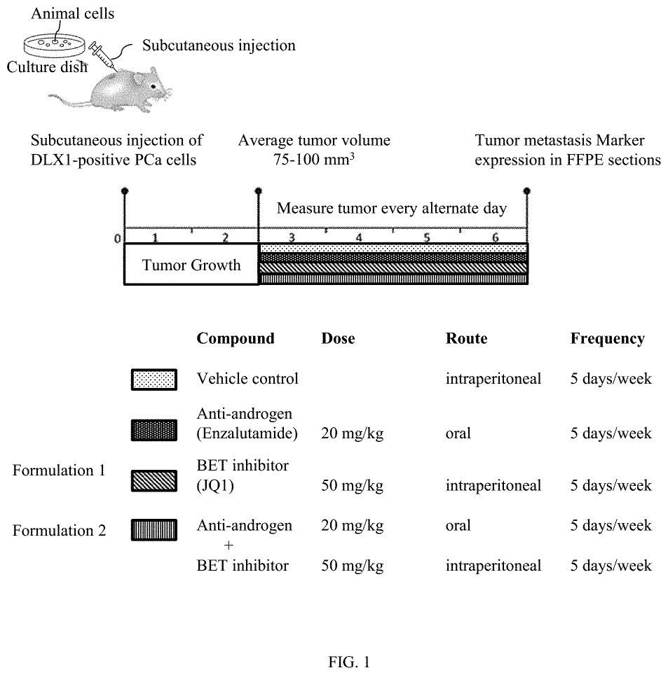

FIG. 1 represents a chart of dosage, route, and frequency of administrating the formulation and a vehicle control, in accordance with an embodiment of the present disclosure.

In an exemplary embodiment, the therapeutically effective amount of the formulation i.e., 50 mg/kg (50 mg compound/kg body weight of mice) of BETi is injected intraperitoneally 5 days a week for total duration of 4 weeks in immunodeficient mice bearing DLX1-positive tumor.

In another exemplary embodiment, the therapeutically effective amount of the formulation i.e., 50 mg/kg of the BETi injected intraperitoneally and 20 mg/kg (20 mg compound/kg body weight of mice) of the anti-androgen drug administered orally 5 days a week for total duration of 4 weeks in immunodeficient mice bearing DLX1-positive tumor.

The therapeutically effective amount of the formulation refers to dosage of the formulation which can effectively reduce the number of cancer cells in the treated subject with significantly minimal or no toxicity. The dosage of the formulation along with frequency and duration of treatment are interdependent and are also influenced by the route of administration and severity of the disease.

In an exemplary embodiment, the method includes administering the therapeutically effective amount of the formulation including only BETi to the subject with prostate cancer at step 102. In another exemplary embodiment, the method includes administering the therapeutically effective amount of the formulation including the combination of BETi and anti-androgen drug to the subject with prostate cancer at step 102.

The formulation (pharmaceutical composition), dosage, frequency, duration and the route of administration are mentioned for preclinical xenograft mice model. They can be accordingly extrapolated and modified for human subjects from the animal models by the skilled artisans.

As used herein the term “bromodomain and extra-terminal domain inhibitors (BETi)” refers to proteins belong to BRD proteins family and share a common domain architecture comprising two N-terminal bromodomains (BD1 and BD2) that interact with acetylated lysine residues on histone tails and an extra-C terminal domain.

The formulation is configured to disrupt E26 oncogene homolog and androgen receptor (ERG/AR) transcriptional circuitry to attenuate Distal-less homeobox gene 1 (DLX1) mediated tumorigenesis. The formulation is also configured to reduce DLX1 expression and downstream target genes of DLX1 in transmembrane protease Serine 2 and ERG (TMPRSS2-ERG) fusion positive prostate cancer cells and TMPRSS2-ERG fusion negative prostate cancer cells. The formulation yields 60% of tumor regression in the subject with prostate cancer.

In the present invention experiments are carried out to understand the relation between DLX1 and PCa. It is shown that higher expression of DLX1 is associated with regulating the oncogenic potential of PCa cells. The TCGA-PRAD data and other RNA-seq datasets identified the upregulation of DLX1 in PCa patients as compared to the adjacent normal tissue or benign prostate tissues. RNA-in situ hybridization for DLX1 in primary and metastatic PCa specimens showed higher expression of DLX1 in ˜60% of the subject with prostate cancer.

In one embodiment, few experiments are carried to demonstrate the role of DLX1 in regulating biological processes including cell proliferation, cell migration, apoptosis, epithelial-to-mesenchymal transition, apoptosis and cancer sternness. These processes play important role during PCa progression.

In one embodiment, control and DLX1-ablated PCa cells are implanted in immunodeficient mice, and reduced tumor growth and metastasis is noted in mice implanted with DLX1 ablated cells. Tumor tissue excised from xenografted mice showed decreased expression of proliferation marker ki-67, DLX1 target ALDH1A1 and mesenchymal marker Vimentin with increased expression of epithelial marker E-cadherin.

In one embodiment, intramedullary tibial injection of control and DLX1-ablated PCa cells followed by microcomputed tomography (microCT), showed higher bone loss and destruction of bone architecture in control group compared to DLX1-ablated group.

TMPRSS2-ERG gene fusion is recurrent in −50% of the primary PCa patients, experiments conducted in the present invention demonstrate that −96% of PCa which shows ERG expression are also positive for DLX1 expression. Additionally, −70% of the AR positive patients, have DLX1 expression implicating towards the possible association of DLX1 with ERG and AR which are the important regulatory factors during PCa progression.

Few experiments shows that ERG directly gets recruited on the upstream 1 kb promoter region of DLX1 thereby regulating its expression in TMPRSS2-ERG positive cases. Few experiments reveal that AR and FOXA1 binds at the enhancer region of DLX1 and contributes to regulating its expression.

The experiments conducted showed the interaction between promoter-bound ERG and enhancer-bound AR and FOXA1 at DLX1 gene loci leading to DLX1 upregulation in TMPRSS2-ERG fusion positive PCa.

In one embodiment, it is depicted that in absence of ERG transcription factor, AR and FOXA1 gets recruited at the DLX1 enhancer region and regulates DLX1 expression. The splice variant AR-V7 functions in a ligand-independent manner and remains constitutively active driving androgen-independent growth and disease progression to CRPC. The findings implicate the role of AR-V7 in regulating DLX1 expression which resonates with the relatively high expression of DLX1 in advanced stage disease.

In the present invention, the efficacy of BETi and anti-androgen drug to target DLX1-positive PCa is discovered.

The experiments demonstrated that BETi may be used to disrupt AR transcriptional circuitry which regulates DLX1 expression in an ERG-independent background while in ERG-dependent context combinatorial treatment of BETi and anti-androgen drug is more effective to attenuate DLX1 expression and its mediated tumorigenesis.

In one embodiment, the formulation has been shown to reduces DLX1 expression and its downstream target genes in both TMPRSS2-ERG fusion-positive and -negative PCa cells.

In an exemplary embodiment, experiment indicates that combination of BETi and anti-androgen drug shows relatively enhanced effect than BETi alone in TMPRSS2-ERG fusion-positive background compared to a fusion-negative background.

In concordance with the in vitro experiments, −60% tumor regression is shown in the mice treated with the BETi and the combination of BETi and anti-androgen drug as compared to the vehicle control mice. Also, the experiments show decrease in the number of cells metastasized to distant organs including lungs and bones in the mice group treated with the formulation as compared to the control group.

In preclinical mice xenograft study, it is discovered that, the formulation led to decreased DLX1 expression, its target genes and DLX1-mediated tumorigenesis thereby demonstrating efficacious treatment regimens with BETi or/and AR targeted therapeutics for the clinical management of DLX1-positive PCa subtype.

The experiments carried in the present invention for evaluation of the formulation in reduction of the DLX1 expression, its downstream target genes and oncogenic properties including cell proliferation, and migration in order to mitigate the prostate cancer are given in a form of examples, which are only by way of illustration and are not to be construed as limiting the scope of the invention.

### EXAMPLES

### Example 1: DLX1 is Upregulated in Prostate Cancer

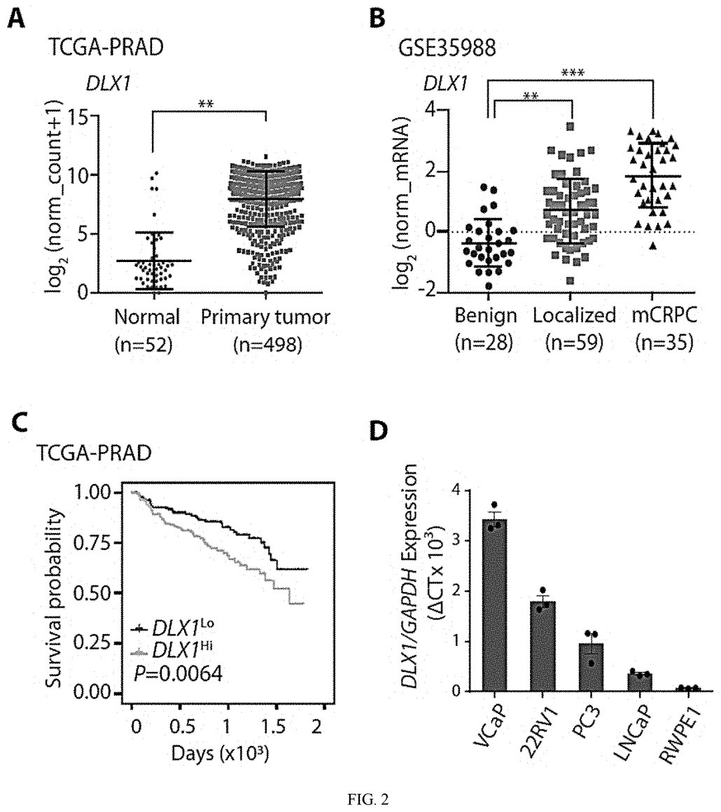

TCGA-PRAD dataset is used to identify expression of DLX1 in primary PCa patients (shown in FIG. 2A).
Clinical genomics datasets (GSE35988) retrieved from gene expression omnibus (GEO) is used to determine expression of DLX1 transcript in advanced stage aggressive cancers compared to benign (shown in FIG. 2B).
Kaplan-Meir survival analysis is performed to investigate role of DLX1 in patients' overall survival (shown in FIG. 2C).
Quantitative-PCR is used to investigate the level of DLX1 expression in PCa cell lines including 22RV1, VCaP, and PC3 that represent CRPC, LNCaP an androgen-responsive cells and RWPE1, a benign and immortalized prostate epithelial cell line (shown in FIG. 2D).

FIG. 2 illustrates upregulation of DLX1 in prostate cancer (PCa) A) Dot plot showing DLX1 expression in TCGA-PRAD RNA-Seq dataset, data represents log 2 (norm_count+1) and whiskers (error bars) denotes standard deviations (SD); B) Dot plot of DLX1 expression using microarray profiling data (GSE35988), log 2 (norm_mRNA), and the error bar represents SD; C) Kaplan-Meier plot showing survival probability in TCGA-PRAD (n=498) dataset categorized in high DLX1 (DLX1^{Hi}) and low DLX1 (DLX1^{Lo}) expression; and D) Q-PCR data showing DLX1 expression in a panel of PCa cell lines, in accordance with an embodiment of the present disclosure.

### Example 2. Characterization of DLX1 Overexpression and Knockout Cells

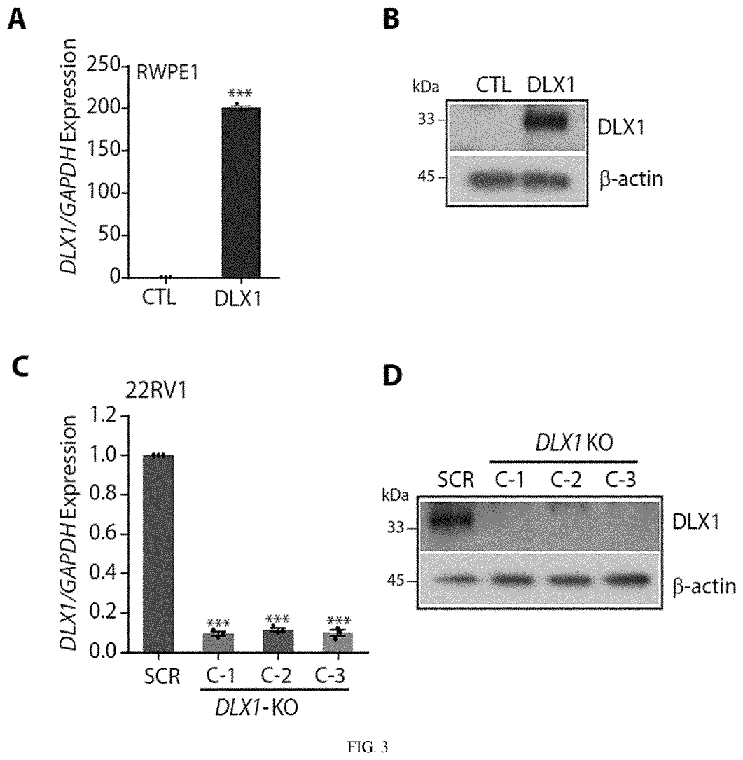

DLX1 is ectopically overexpressed in RWPE1 cells and expression is confirmed at transcript level using q-PCR (shown in FIG. 3A).
Increase in DLX1 expression is confirmed at protein level (shown in FIG. 3B). CRISPR/Cas9 mediated knockout of DLX1 is performed in 22RV1 prostate cancer cell line and expression of DLX1 in three independent clones is confirmed at transcript level (shown in FIG. 3C).
Decrease in DLX1 expression is confirmed at protein level (shown in FIG. 3D).

FIG. 3 illustrates characterization of DLX1 overexpression and knockout cells A) Q-PCR data showing relative expression of DLX1 in isogenic RWPE1 cells overexpressing DLX1; B) Immunoblot showing protein level of DLX1 in cells same as A); C) Q-PCR showing DLX1 expression in 22RV1-DLX1-KO and 22RV1-SCR control cells; and D) Immunoblot showing protein level of DLX1 in cells same as C), in accordance with an embodiment of the present disclosure.

### Example 3. DLX1 Regulates Oncogenic Properties in Prostate Cells

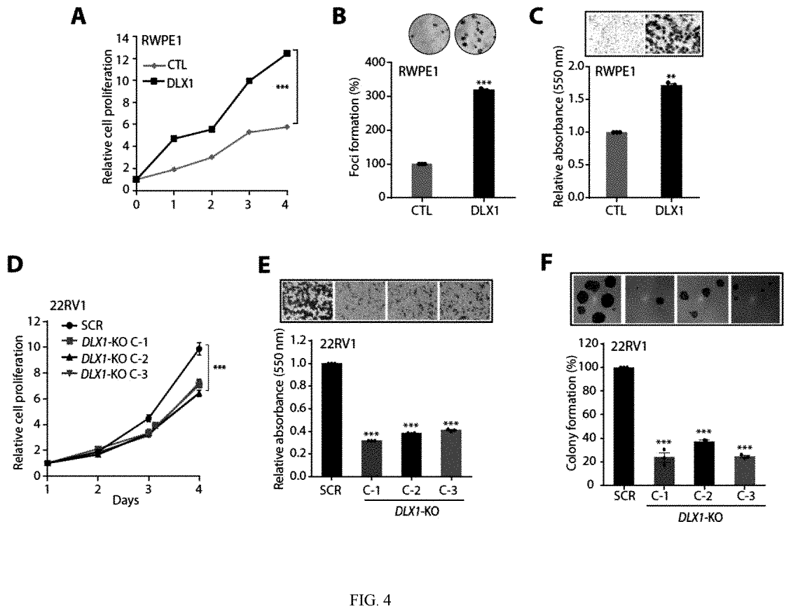

Relative cell proliferation rate of RWPE1-CTL and RWPE1-DLX1 overexpressing cells is examined by counting cells in both the conditions for four consecutive days (shown in FIG. 4A).
Foci formation assay is performed using RWPE1-CTL and RWPE1-DLX1 overexpressing cells and cells are grown in reduced serum conditions. Foci are counted at the end of experiments (shown in FIG. 4B).
Cell migration assay is performed using RWPE1-CTL and RWPE1-DLX1 overexpressing cells in Transwell Boyden chamber and migrated cells are quantified after 24 hrs. (shown in FIG. 4C).
Relative cell proliferation rate of 22RV1-SCR and 22RV1-DLX1-KO cells is examined by counting cells in both the conditions for four consecutive days (shown in FIG. 4D).
Foci formation assay is performed using 22RV1-SCR and DLX1-KO cells. Cells are grown in reduced serum conditions. Foci are counted at the end of experiments (shown in FIG. 4E).
Anchorage-independent colony formation assay is performed using 22RV1-SCR and DLX1-KO cells. Colonies are counted at the end of the experiment (shown in FIG. 4F).

FIG. 4 illustrates DLX1 regulates oncogenic properties in prostate cells A) cell proliferation assay using isogenic RWPE1 cells overexpressing DLX1 at indicated time-points; B) foci formation assay using same cells as A); C) Boyden Chamber Matrigel migration assay using same cells as A); D) cell proliferation assay using 22RV1-DLX1-KO (C-1, C-2 and C-3 are independent clones) and control cells at indicated time-points; E) Boyden Chamber Matrigel migration assay using same cells as D); and F) Anchorage-independent soft agar assay using same cells as D), in accordance with an embodiment of the present disclosure. Representative images for panels (E) and (F) are shown as inset.

### Example 4. DLX1 Regulates Tumor Associated Biological Processes

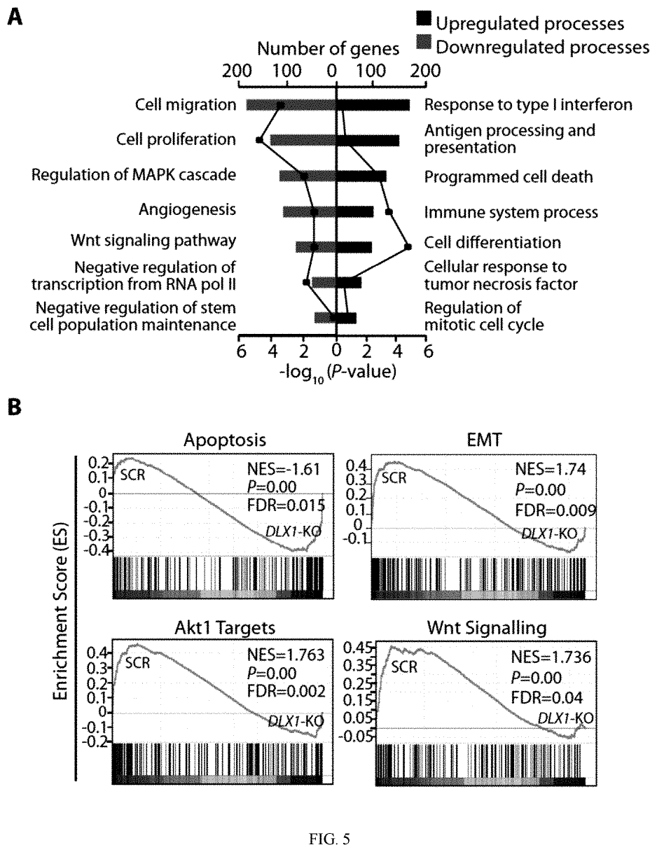

Database for Annotation, Visualization and Integrated Discovery (DAVID, https://david.ncifcrf.gov/) bioinformatics analysis on the differentially expressed genes identified using global gene expression profiling of 22RV1-SCR control and DLX1-KO cells (shown in FIG. 5A).
Gene set enrichment analysis on the differentially expressed genes (shown in FIG. 5B).

FIG. 5 illustrates regulation of tumor associated biological processes by DLX1 A) DAVID analysis showing unregulated (right) and downregulated (left) biological processes in 22RV1-DLX1-KO against control cells, bars represent the −log 10 (P-value) and the frequency polygon (black line) denotes number of genes; and B) gene set enrichment analysis (GSEA) plots representing deregulated pathways, in accordance with an embodiment of the present disclosure.

### Example 5. Genetic Ablation of DLX1 Orchestrates Cancer Hallmarks

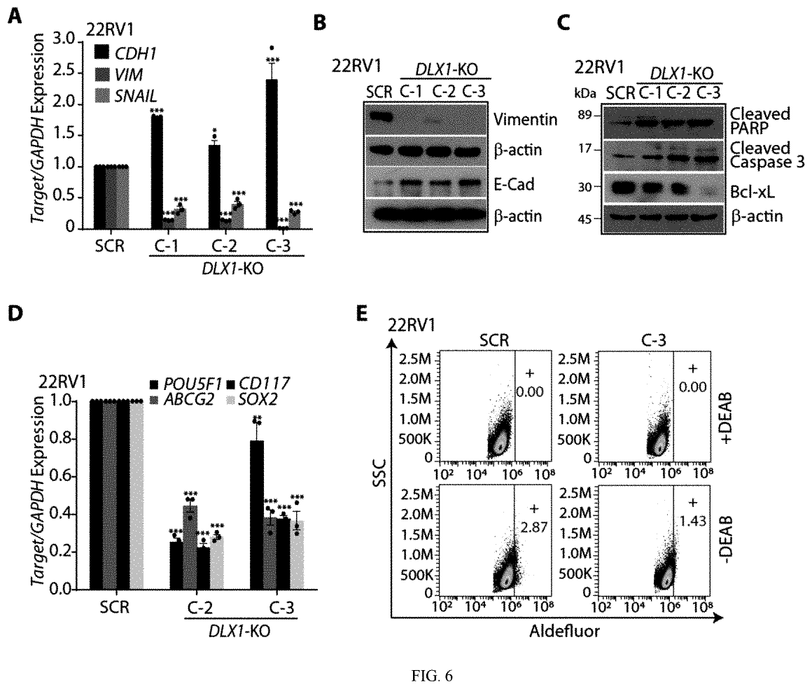

Expression of EMT markers is explored at transcript level and protein level using q-PCR and immunoblot in 22RV1-SCR control and DLX1-KO cells (shown in FIGS. 6A and 6B).
Immunoblot is performed to investigate the protein level of apoptotic markers, namely, cleaved Poly (ADP-ribose) polymerase (PARP), cleaved caspase 3, and Bcl-xL (shown in FIG. 6C).
Cancer stem cell markers, namely, POU5F1 (Oct-4), CD117 (c-Kit), ABCG2 and SOX2 are analysed at transcript level by performing q-PCR in 22RV1-SCR control and DLX1-KO cells (shown in FIG. 6D).
Aldefluor assay is performed to determine aldehyde dehydrogenase (ALDH) enzymatic activity using flow cytometry. ALDH inhibitor, diethylamino benzaldehyde (DEAB) is used as a negative control to identify cells showing ALDH activity (shown in FIG. 6E).

FIG. 6 illustrates genetic ablation of DLX1 orchestrates cancer hallmarks A) Q-PCR data showing expression of EMT markers in 22RV1-DLX1-KO and control SCR cells; B) immunoblots showing vimentin and E-cadherin using same cells as A), β-actin is used as loading control; C) immunoblots showing vimentin and E-cadherin same as B) except for cleaved PARP, cleaved Caspase-3 and Bcl-xL; D) Q-PCR data for stem cell markers using 22RV1-DLX1-KO and control SCR cells; and E) fluorescence intensity of catalyzed ALDH substrate in 22RV1-DLX1-KO and control cells, marked windows show ALDH1+ percent cell population, in accordance with an embodiment of the present disclosure.

### Example 6. Abrogating DLX1 Expression Results in Tumor Regression and Reduced Metastases

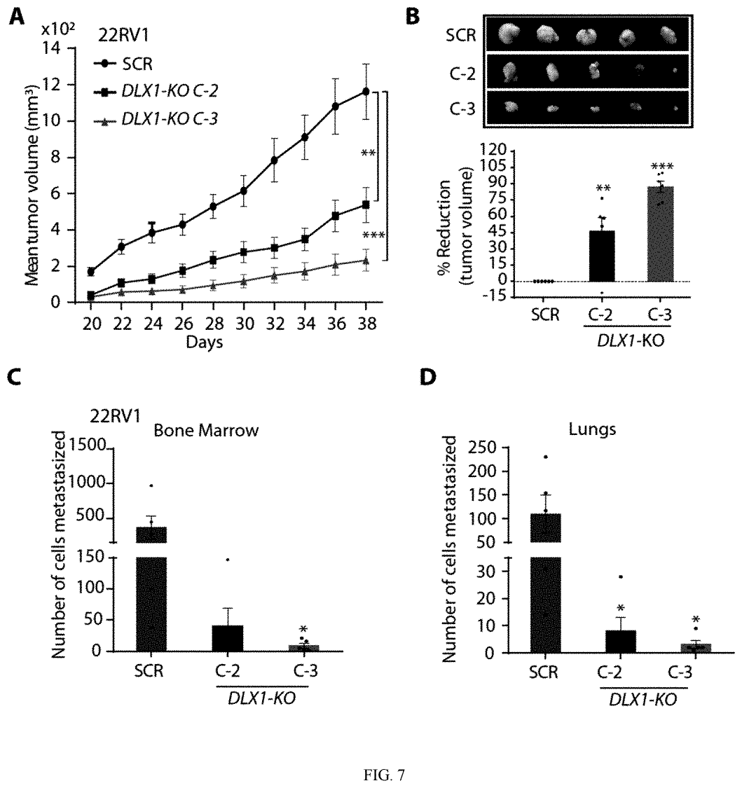

22RV1-SCR and DLX1-KO cells are subcutaneously implanted on the flank region of five to six weeks old immunodeficient male mice and tumor burden is measured every alternate day (shown in FIGS. 7A and 7B).
Bone marrow and lungs of the xenografted mice are excised at the end of the study, genomic DNA is isolated and is analyzed for the presence of human Alu-sequences by performing Taqman assay (shown in FIGS. 7C and 7D).

FIG. 7 illustrates DLX1 expression abrogating results in tumor regression and reduced metastases A) mean tumor volume of 22RV1-DLX1-KO and control SCR cells subcutaneously implanted in NOD/SCID mice (n=6); B) representative images of the tumors excised at end of the xenograft experiment (top panel), bar graph showing relative percent reduction in tumor burden (bottom panel); C) bar graphs representing number of cells metastasized to the bone marrow in xenografted mice as labelled (n=5); and D) bar graphs representing number of cells metastasized to the lungs in xenografted mice as labelled (n=5), in accordance with an embodiment of the present disclosure.

### Example 7. DLX1 Attenuation Regulates Proliferation, EMT and Sternness Markers

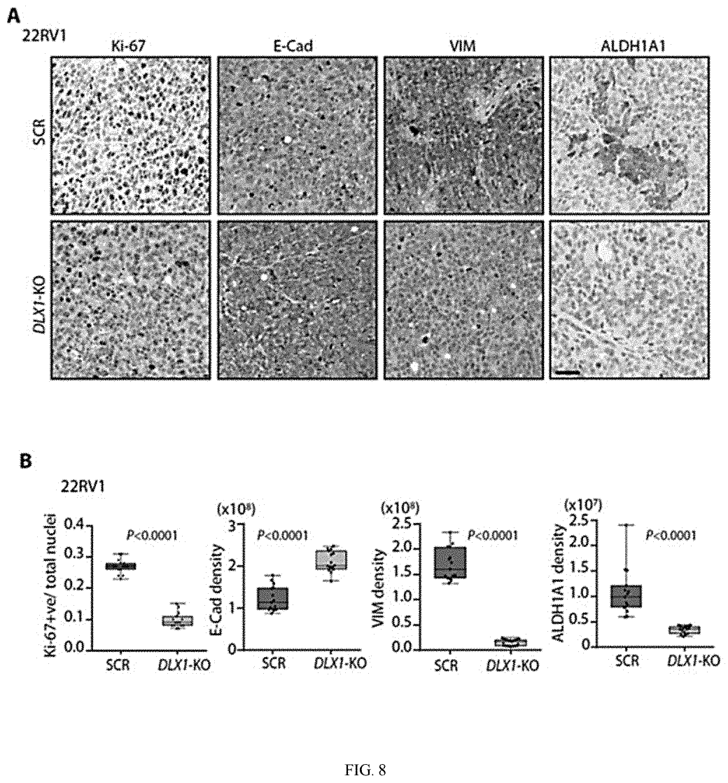

Immunohistochemistry (IHC) is performed for Ki-67 (proliferation marker), E-Cad and vimentin (EMT markers) and ALDH1A1 (stemness marker and DLX1 target gene) on the formalin-fixed paraffin-embedded (FFPE) tumor tissue excised from xenografted mice (shown in FIG. 8A).
Quantification of the stained sections is performed using ImageJ software (shown in FIG. 8B).

FIG. 8 illustrates regulation of DLX1 attenuation proliferation, EMT and stemness markers A) images depicting immunostaining for Ki-67, E-cadherin (E-Cad), Vimentin (VIM), and ALDH1A1 on xenograft tumor sections; B) box plots showing immunostaining quantification of Ki-67, E-Cad, VIM and ALDH1A1, in accordance with an embodiment of the present disclosure. Images in A) are representative of 3 tissue samples. Scale bar of B) is 35 μm and quantification is blindly done from 15 random histological fields.

### Example 8. DLX1 Regulates the Bone Metastatic Potential of PCa Cells

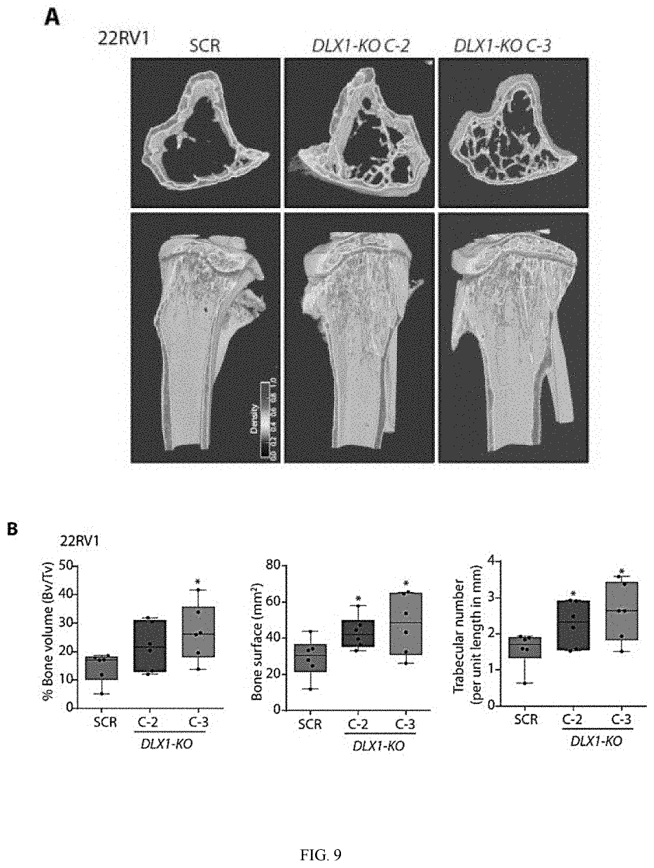

Athymic nude (NU(NCr)-Foxn1nu), 5-6-week-old male mice are anesthetized. 22RV1-SCR or DLX1-KO cells are implanted by intramedullary tibial injections using 26-gauge needle. After 4 weeks, mice are subjected to X-ray scan and are euthanized. Tibia subjected to injections are harvested and analyzed using micro-CT. 3D image reconstruction and visualization are performed using volume rendering program CTVox (shown in FIG. 9A).
The analysis of the bone lesions and bone morphometric parameters is performed using microCT. The CTAn v1.16.8.0 software is used for 3D image processing and parametric analysis (shown in FIG. 9B).

FIG. 9 illustrates regulation of the bone metastatic potential of PCa cells by DLX1 A) representative microCT bone images showing horizontal section (top panel) and vertical cross-section (bottom panel) views of the tibia excised from mice (n=6), four weeks after intra-medullary tibia injection using 22RV1-DLX1-KO and control cells; B) representative microCT bone images same as A) except box plots showing bone architecture parameters analyzed using CTAn software, in accordance with an embodiment of the present disclosure.

### Example 9. Elevated ERG and AR Expression Correlates with Higher DLX1 Levels

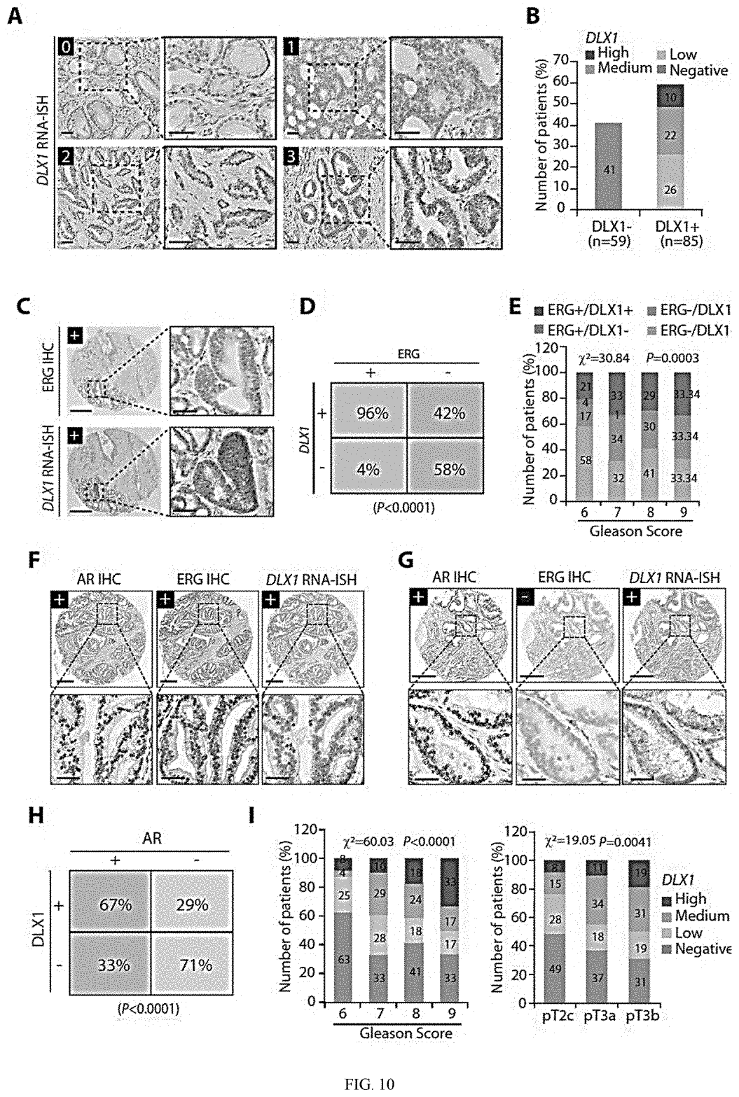

DLX1 expression is identified by scoring the signal intensity of RNA-ISH probe hybridization for the TMA foci (n=144) and the number of red dots/cell are evaluated to grade DLX1 expression into four levels ranging from score of 0 to 3 as negative to high (shown in FIG. 10A).
Samples are analysed for DLX1 expression and the specimens with score 0 are considered as DLX1-negative while patients with score 1, 2, and 3 are considered DLX1-positive (shown in FIG. 10B).
The TMA samples are compared for the association between ERG and DLX1 expression. Contingency table is plotted to compare the DLX1-positive and-negative percent patients with respect to ERG-positive and -negative patients (shown in FIGS. 10C and 10D).
The clinical characteristics of the patients are compared with ERG and DLX1 expression in the patients. The bar plot demonstrates the association of Gleason score with the ERG and DLX1 positive/negative patients (FIG. 9E).
IHC is performed for AR and ERG, RNA-ISH is performed for DLX1 on tissue microarray comprising of tumor tissues from 144 primary prostate cancer patients (shown in FIGS. 10F and 10G).
Contingency table representing the percent of patients positive for DLX1 expression with respect to the patients showing positive and negative expression of AR (shown in FIG. 10H).
Analysis of DLX1 expression with respect to clinical characteristics including Gleason scoring and tumor staging is performed (shown in FIG. 10I).

FIG. 10 illustrates correlation of elevated ERG and AR expression with higher DLX1 levels A) representative core of the PCa tissue microarray (TMA) showing RNA in-situ hybridization (RNA-ISH) scoring pattern for DLX1 in 144 PCa patient specimens; B) bar plot showing percentage of patients negative (DLX1−) and positive (DLX1+) for DLX1 expression based on the scoring pattern; C) representative core of the PCa TMA same as A) except Immunohistochemistry (IHC) for ERG (top panel) and RNA-ISH for DLX1 (bottom) in 144 PCa patient tissue specimens; D) contingency table depicting status of DLX1 and ERG; E) bar plot showing association between ERG and DLX1 expression status and Gleason scores of PCa patients; F) representative core of the PCa TMA same as A) except representative tumor cores showing IHC for AR, ERG and RNA-ISH for DLX1 representing AR+/ERG+/DLX1+ status in 144 PCa patient tissue specimens; G) same as F) except for representative AR+/ERG−/DLX1+ patient in TMA containing 144 PCa specimens; H) contingency table for the AR and DLX1 status in TMA patient specimens; and I) bar plot showing association between DLX1 expression and Gleason scores (left panel) of tumor specimens, and association of DLX1 expression with tumor stage (right panel), in accordance with an embodiment of the present disclosure. In A), score 0 represents DLX1 negative, score 1 signifies low DLX1, score 2 and score 3 represents medium and high DLX1 expression, respectively. Scale bar of A) is 50 μm. In D), pink panel shows status of DLX1 patients in ERG positive cases (left) and blue panel shows status of DLX1 in ERG negative cases (right). P-value for Fisher's exact test is indicated. In H), P-value denotes Fisher's exact test.

### Example 10. Hither Level of DLX1 in Metastatic CRPC Patients

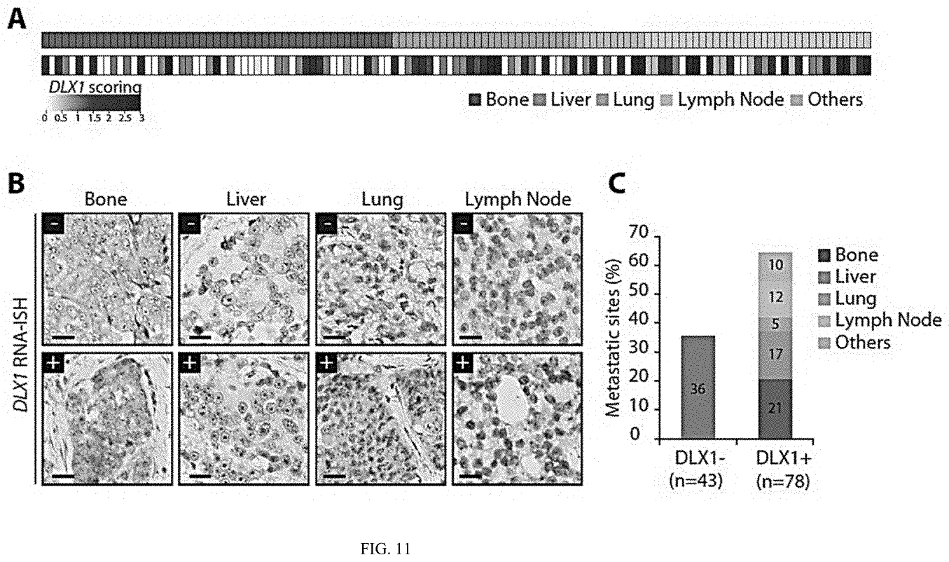

TMA comprising of 121 biospecimens obtained from different metastatic sites from patients died with CRPC is used to perform RNA-ISH for DLX1. Percent of metastatic sites positive for DLX1 expression are identified and grouped according to the tissue types (shown in FIG. 11).

FIG. 11 illustrates higher level of DLX1 in metastatic CRPC patients A) Heatmap showing DLX1 levels in tumor specimens representing distant metastatic sites of metastatic CRPC patients; B) Heatmap showing DLX1 levels same as A) except for RNA-ISH for DLX1 expression in TMA containing 121 mCRPC biospecimens collected from various metastatic sites; and (C) bar plot showing DLX1 expression in percent metastatic sites from CRPC patients same as A), in accordance with an embodiment of the present disclosure. In C), scale bar is 25 μm.

### Example 11. DLX1 is a Transcriptional Target of ERG and/or AR in PCa

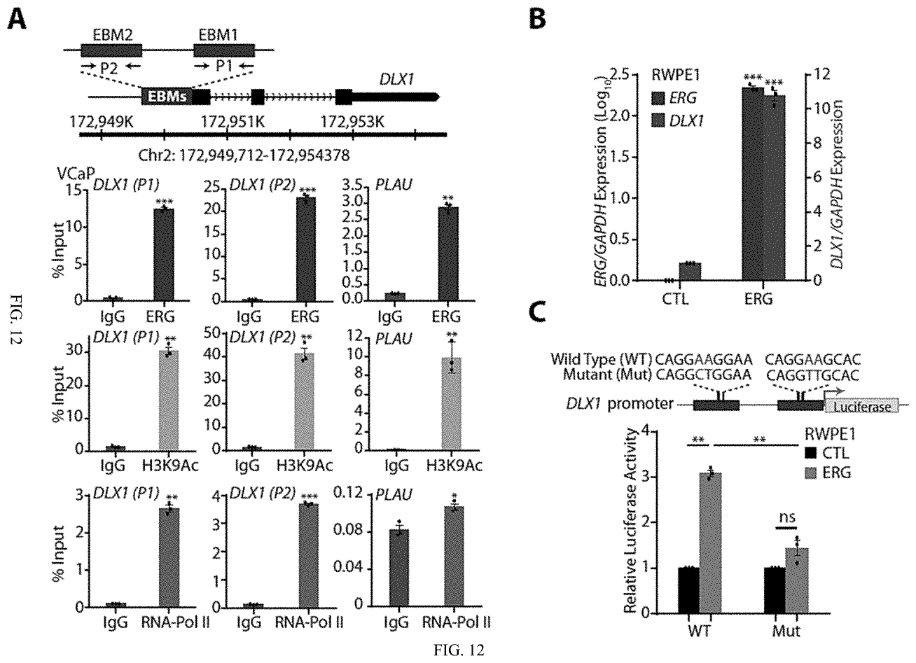

Chromatin immunoprecipitation followed by q-PCR (ChIP-PCR) is performed using antibody against ERG, H3K9Ac and RNA-Pol II in VCaP (ERG-positive) cells. Cells are crosslinked and are then lysed and DNA is fragmented. Sheared chromatin is incubated with respective antibodies and Protein G coated Dynabeads. The beads conjugated with antibody are washed and immunocomplex is precipitated and eluted. DNA is isolated and PCR is performed (shown in FIG. 12A).
ERG is ectopically overexpressed in RWPE1 cells and expression of ERG and DLX1 is investigated at transcript level (shown in FIG. 12B).
Site directed mutagenesis is performed to mutate the ERG binding motif in DLX1 promoter region. Luciferase reporter is performed in RWPE1-CTL and RWPE1-ERG overexpressing cells using wildtype (WT) and mutated (Mut) DLX1 promoter. Firefly and Renilla luciferase activity are measured using the luminometer (shown in FIG. 12C).

FIG. 12 illustrates DLX1 is a transcriptional target of ERG and/or AR in PCa A) schema showing chromosomal location of the ERG binding motifs (EBM) onto DLX1 promoter selected for ChIP-qPCR (top panel). ChIP-qPCR data showing recruitment of ERG at the DLX1 promoters; B) Q-PCR data showing the expression of ERG and DLX1 in RWPE1 cells overexpressing ERG; and C) schema showing site-directed mutagenesis of DLX1 promoter cloned upstream of luciferase gene, nucleotides in red are mutated (top), in accordance with an embodiment of the present disclosure. Luciferase reporter assay indicating wild-type (WT) and mutant (Mut) DLX1 promoter-driven reporter activity (bottom panel) in ERG overexpressing and control RWPE1 cells.

### Example 12. ERG, AR and FOXA1 Coordinates to Regulate DLX1 Expression in TMPRSS2-ERG Fusion Positive PCa

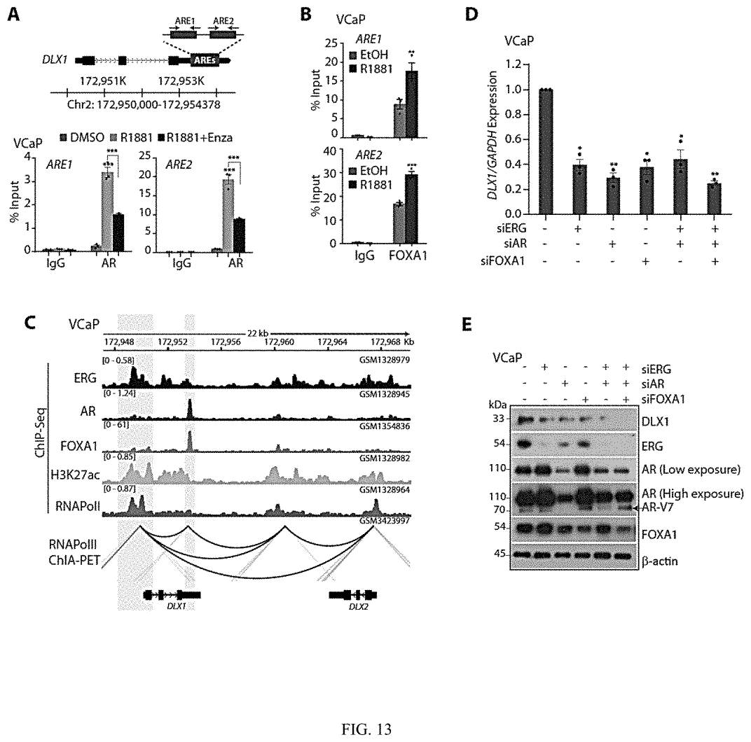

ChIP-PCR is performed using AR antibody in VCaP cells. AR recruitment at DLX1 enhancer is investigated in R1881 stimulated and anti-androgen treated cells (shown in FIG. 13A).
ChIP-PCR is performed using antibody against FOXA1 in R1881 stimulated VCaP cells (shown in FIG. 13B).
3D-chromatin landscape of RNA-Pol II in VCaP cells using ChIA-PET dataset (GSE121020) is analysed. The integrative analysis of RNA-Pol II associated peaks along with ChIP-Seq data of DLX1 regulating TFs in PCa is performed (shown in FIG. 13C).
Transient knockdown is performed in VCaP cells using small-interfering RNA against ERG, AR and FOXA1 alone or in combination. DLX1 expression is investigated at the transcript and protein level (shown in FIGS. 13D and 13E).

FIG. 13 illustrates regulation of DLX1 expression in TMPRSS2-ERG fusion positive PCa by coordinating ERG, AR and FOXA1 coordinates A) schematic showing the androgen response elements (AREs) at the DLX1 putative enhancer (top panel); B) ChIP-qPCR data depicting FOXA1 recruitment at the DLX1 putative enhancer in R1881 (10 nM) stimulated VCaP; C) integrated genome view of 3D chromatin structure and binding of transcription factors at the genomic and nearby region of DLX1; D) Q-PCR data showing relative expression of DLX1 in siRNA-mediated ERG, AR and/or FOXA1 silenced VCaP cells; and E) same as D) except immunoblot data, in accordance with an embodiment of the present disclosure. In A), ChIP-qPCR data depicts AR recruitment at the DLX1 putative enhancer in R1881 (10 nM) stimulated VCaP cells in the presence or absence of Enzalutamide (Enza, 10 μM) (bottom panel).

### Example 13. AR and FOXA1 Regulates Transcriptional Regulation of DLX1 in TMPRSS2-ERG Fusion Positive PCa

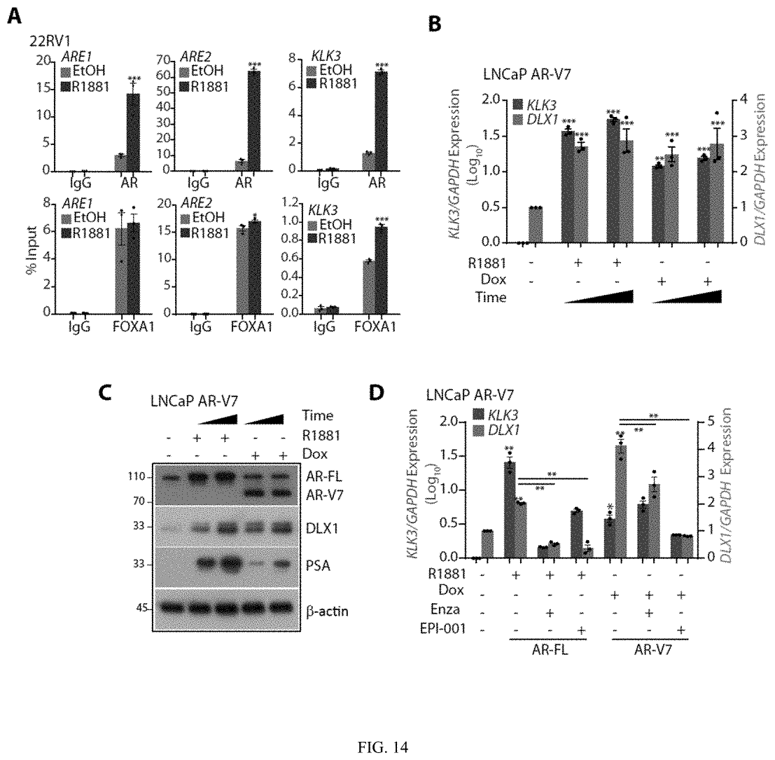

ChIP-PCR is performed using AR antibody to investigate its occupancy on DLX1 enhancer region in TMPRSS2-ERG negative PCa cells (shown in FIG. 14A).
Tetracycline inducible AR-V7 overexpressing LNCaP cells are established. Cells are treated with R1881 or with doxycycline (dox) to induce AR-FL or AR-V7 expression in LNCaP cells, respectively. Expression of DLX1, PSA and AR are investigated at transcript and protein level (shown in FIGS. 14B and 14C).
LNCaP AR-V7 cells are treated with AR antagonists (enzalutamide or EPI-001) in the presence of R1881 or dox and change in DLX1 and KLK3 expression are investigated at transcript level (shown in FIG. 14D).

FIG. 14 illustrates regulation of transcriptional regulation of DLX1 in TMPRSS2-ERG fusion positive PCa by AR and FOXA1 A) ChIP-qPCR data depicting AR (top panel) and FOXA1 (bottom panel) enrichment at the DLX1 putative enhancer in 22RV1 cells stimulated with R1881 (10 nM) for 16 hours; B) relative expression of KLK3 and DLX1 in doxycycline (Dox) induced AR-V7 overexpressing LNCaP cells treated with R1881 (10 nM); C) immunoblots showing the expression of AR-FL, AR-V7, DLX1 and PSA using same cells as B); D) Q-PCR data showing DLX1 and KLK3 expression in LNCaP AR-V7 cells under similar culture conditions as mentioned at 48-hour time point; in accordance with an embodiment of the present disclosure. In A), KLK3 shown as a positive control. In B), for induction 40 ng/ml of Dox or vehicle control is used for 24 hours and 48 hours. In C), β-actin used as loading control.

### Example 14. BET Inhibitor Alone or in Combination with Anti-Androgen Downregulates DLX1 Expression

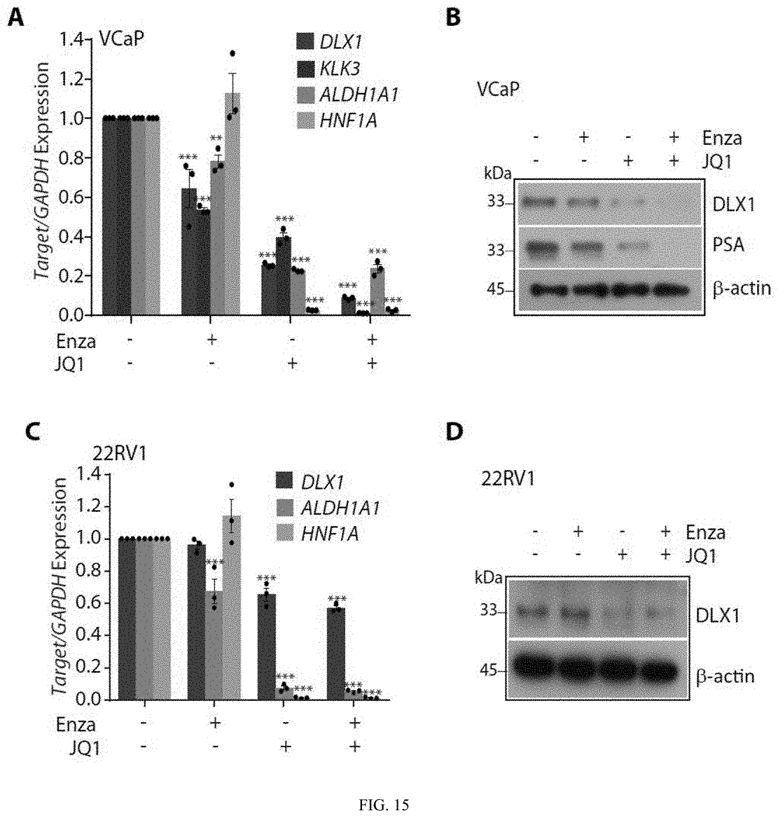

VCaP (TMPRSS2-ERG positive) cells are treated with JQ1 (BETi), Enzalutamide (anti-androgen) alone or in combination and q-PCR are performed to investigate the effect on DLX1 expression and its target genes (shown FIGS. 15A and 15B).
22RV1 (TMPRSS2-ERG negative) cells are treated with JQ1 (BETi) and Enzalutamide (anti-androgen) and q-PCR is performed to investigate the effect on DLX1 expression and its target genes (shown in FIGS. 15C and 15D).

FIG. 15 illustrates downregulation of DLX1 expression by BET inhibitor alone or in combination with anti-androgen A) Q-PCR data showing relative expression of DLX1, KLK3 and DLX1 target genes, namely, HNF1A and ALDH1A1 in VCaP cells treated with Enza (10 μM), JQ1 (0.5 μM) alone or in combination for 48 hours; B) Q-PCR data same as A) except immunoblot; C) Q-PCR data same as A) except 22RV1 cells; and D) Q-PCR data same as C) except immunoblot, in accordance with an embodiment of the present disclosure. In A), KLK3 used as a positive control for JQ1 treatment. In B), β-actin is used as loading control.

### Example 15. BET Inhibitor or/and Anti-Androgen Orchestrates DLX1 Mediated Oncogenic Properties

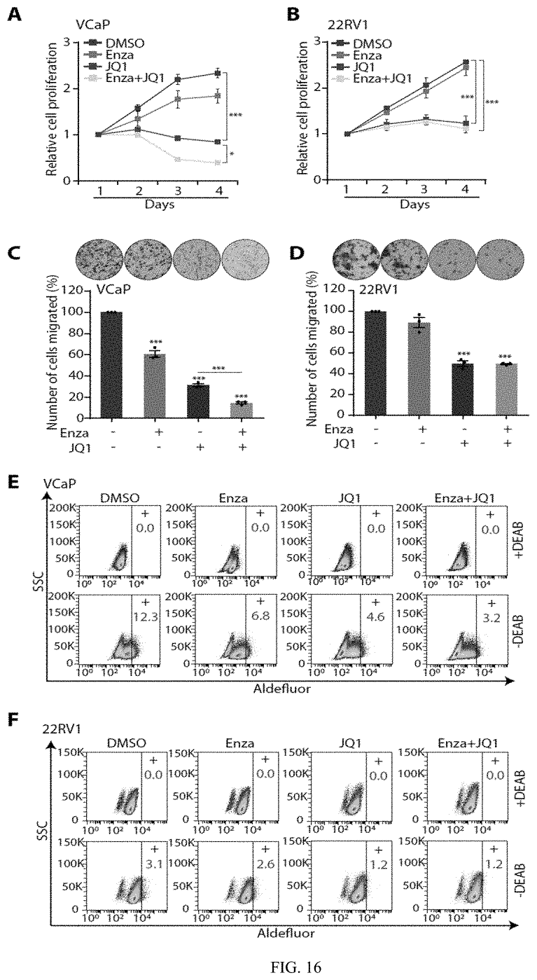

Cell proliferation assay is performed in VCaP and 22RV1 cells after drug (the formulation) treatment and cells are counted every 24 hours for four days (shown in FIGS. 16A and 16B).
Boyden Chamber Matrigel assay is performed to investigate the effect of drug (the formulation) treatment on the migratory properties of cells (shown in FIGS. 16C and 16D).
Flow cytometry-based aldefluor assay is performed in VCaP and 22RV1 cells treated with BETi or/and antiandrogen (shown in FIGS. 16 E and 16F).

FIG. 16 illustrates BET inhibitor or/and anti-androgen orchestrates DLX1 mediated oncogenic properties A) cell proliferation assay in VCaP cells treated Enza (10 μM), JQ1 (0.5 μM) alone or in combination for 48 hours; B) cell proliferation assay same as A) except 22RV1 cells; C) Boyden Chamber Matrigel migration assay in VCaP cells using same treatment conditions as A); D) Boyden Chamber Matrigel migration assay same as C) except 22RV1 cells; E) Fluorescence intensity of the catalyzed ALDH substrate in VCaP cells under same treatment conditions as A); and F) Fluorescence intensity same as E) except 22RV1 cells, in accordance with an embodiment of the present disclosure. In C), inset shows representative image of the migrated cells. In E), marked windows show ALDH1+ percent cell population.

### Example 16. BET Inhibitor Alone or in Combination with Anti-Androgen Attenuates DLX1-Mediated Tumorigenesis and Metastases

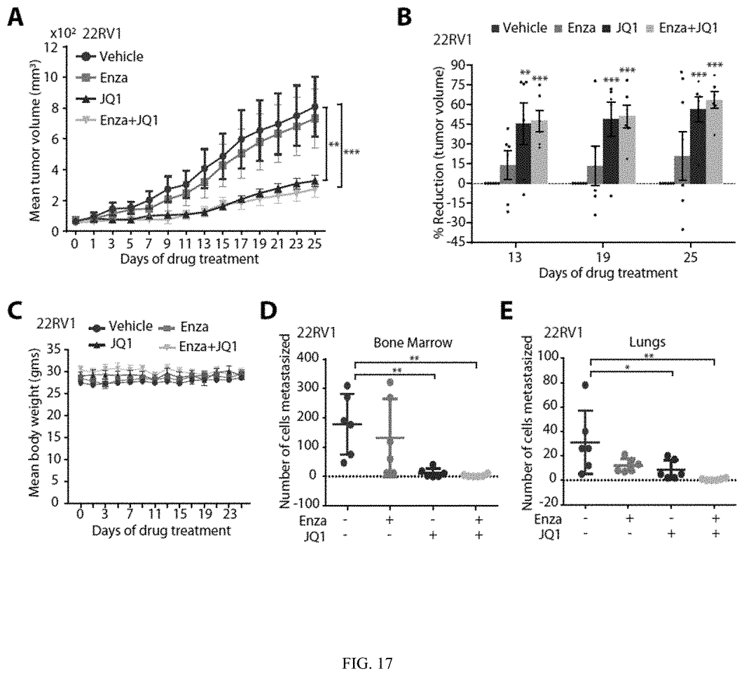

22RV1 cells are subcutaneously implanted in athymic immunodeficient mice, and when the tumors reached a palpable stage (average volume ˜75 mm3); mice are randomized into four groups (n=6) and the drugs (the formulation) are administered (shown in FIGS. 17A and 17B).
Mice body weight is measured every alternate day to study the deleterious effect of drug (the formulation), if any (shown in FIG. 17C).
Bone marrow and lungs of the xenografted mice are excised at the end of the study and analyzed by performing Taqman assay using primers specific for human Alu-sequences (shown in FIGS. 17D and 17E).

FIG. 17 illustrates attenuation of DLX1-mediated tumorigenesis and metastases by BET inhibitor alone or in combination with anti-androgen A) mean tumor volume of xenografts generated by implanting 22RV1 cells in athymic nude mice, and randomized into four treatment groups (n=6 each), namely, vehicle control, Enza (20 mg/kg), JQ1 (50 mg/kg), and a combination of Enza and JQ1; B) bar plot showing percent tumor reduction in the treatment groups (n=6) compared with vehicle control group; C) mean body weight of mice during treatment with drugs (the formulation) as mentioned in A); D) scatter dot plot showing number of cells metastasized to bone marrow in xenografted mice treated with drugs (the formulation) as mentioned in A); and E) scatter dot plot same as D) except for showing number of cells metastasized to lungs, in accordance with an embodiment of the present disclosure. In D), data represent mean±SD.

### Example 17. BET Inhibitor or/and Anti-Androgen Reduces Expression of DLX1, Proliferative and Stem-Cell Marker

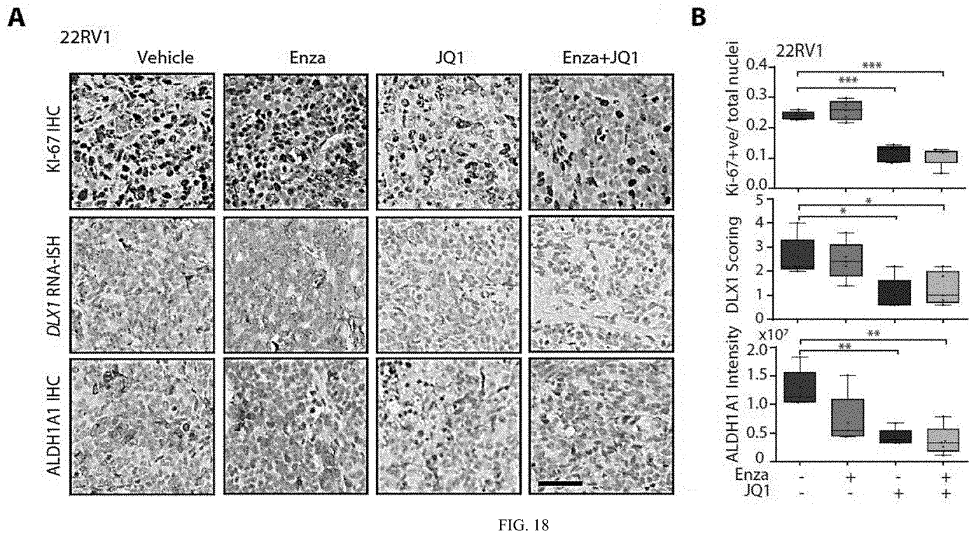

Immunohistochemistry (IHC) is performed for Ki-67 (proliferation marker), ALDH1A1 (stemness marker and DLX1 target gene) and RNA-ISH is performed for DLX1 on the formalin-fixed paraffin-embedded (FFPE) tumor tissue excised from xenografted mice (shown in FIG. 18A).
Quantification of the stained sections is performed using ImageJ software (shown in FIG. 18B).

FIG. 18 illustrates reduction in expression of DLX1 by BET inhibitor or/and anti-androgen, proliferative and stem-cell marker A) representative images depicting IHC staining for Ki-67 (proliferation marker), ALDH1A1 (DLX1 target gene), and RNA-ISH for DLX1 using formalin-fixed paraffin-embedded tumor xenograft specimens of nude mice administered with vehicle control, Enza (20 mg/kg), JQ1 (50 mg/kg), and a combination of Enza and JQ1 (n=5 per group); and B) box plots showing quantification of Ki-67, ALDH1A1 and DLX1 expression in the tumor tissue sections (n=5) of the mice xenografts as A), in accordance with an embodiment of the present disclosure. In A) scale bar is 50 μm. In B), data are presented as box-and-whisker plots indicating median (middle line), 25th and 75th percentile (box) and minimum and maximum values (whiskers).

The present invention provides the formulation and the method for treating prostate cancer. The formulation including BETi and the combination of BETi and anti-androgen drug yields 60% of tumor regression in the prostate cancer patient. The method enables disruption of ERG/AR transcriptional circuitry to attenuate DLX1 mediated tumorigenesis. The method provides administration of the formulation to the subject with prostate cancer. The formulation is configured to reduce DLX1 expression and downstream target genes of DLX1 in TMPRSS2-ERG fusion positive prostate cancer cells and TMPRSS2-ERG fusion negative prostate cancer cells.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A formulation for treating prostate cancer, comprising:
at least one of bromodomain and extra-terminal domain inhibitors (BETi) and an anti-androgen drug or a combination thereof,
wherein the formulation is configured to
disrupt E26 oncogene homolog and androgen receptor (ERG/AR) transcriptional circuitry to attenuate Distal-less homeobox gene 1 (DLX1) mediated tumorigenesis, and
reduce DLX1 expression and downstream target genes of DLX1 in transmembrane protease Serine 2 and ERG (TMPRSS2-ERG) fusion positive prostate cancer cells and TMPRSS2-ERG fusion negative prostate cancer cells.

2. The formulation of claim 1, wherein the BETi comprises JQ1, wherein the JQ1 is selected from a group consisting of BRD4, BRD2, BRD2/4 and a combination thereof.

3. The formulation of claim 1, wherein the formulation comprises the BETi in an amount of 50 mg/kg of body weight of a subject for treating DLX1-positive prostate cancer.

4. The formulation of claim 1, wherein the anti-androgen drug comprises Enzalutamide.

5. The formulation of claim 1, wherein the formulation comprises the BETi in combination with the anti-androgen drug for treating DLX1-positive prostate cancer, wherein the BETi is in an amount of 50 mg/kg of body weight of the subject and the anti-androgen drug in an amount of 20 mg/kg body weight of the subject.

6. The formulation of claim 1, wherein the formulation yields 60% of tumor regression.

7. A method for treating prostate cancer, comprising:
administering a therapeutically effective amount of a formulation to a subject with prostate cancer,
wherein the formulation comprises at least one of bromodomain and extra-terminal domain inhibitors (BETi) and an anti-androgen drug or combination thereof,
wherein the formulation is configured to
disrupt E26 oncogene homolog and androgen receptor (ERG/AR) transcriptional circuitry to attenuate Distal-less homeobox gene 1 (DLX1) mediated tumorigenesis, and
reduce DLX1 expression and downstream target genes of DLX1 in transmembrane protease Serine 2 and ERG (TMPRSS2-ERG) fusion positive prostate cancer cells and TMPRSS2-ERG fusion negative prostate cancer cells.

8. The method of claim 7, wherein the BETi comprises JQ1, wherein the JQ1 is selected from a group consisting of BRD4, BRD2, BRD2/4 and a combination thereof.

9. The method of claim 7, wherein the formulation comprises the BETi in an amount of 50 mg/kg of body weight of a subject for treating DLX1-positive prostate cancer. The method of claim 7, wherein the anti-androgen drug comprises Enzalutamide.

10. The method of claim 7, wherein the formulation comprises the BETi in combination with the anti-androgen drug for treating DLX1-positive prostate cancer, wherein the BETi is in an amount of 50 mg/kg of body weight of the subject and the anti-androgen drug in an amount of 20 mg/kg body weight of the subject.

11. The method of claim 7, wherein the formulation yields 60% of tumor regression.
